(19) United States
(12) Patent Application Publication
(10) Pub. No.: US 2023/0184744 A1
(43) Pub. Date: Jun. 15, 2023

(54) INTERTUMORAL HOMOGENEITY DETERMINED BY MiCK ASSAY

(71) Applicant: Pierian Biosciences, LLC, Smyma, TN (US)

(72) Inventors: Cary Presant, San Marino, CA (US); Mathieu Perree, Montreal (CA); Allan E. Hallquist, Morning View, KY (US)

(21) Appl. No.: 18/078,240

(22) Filed: Dec. 9, 2022

Related U.S. Application Data
(63) Continuation of application No. 16/953,646, filed on Nov. 20, 2020, now abandoned.
Application No. 16/953,646 is a continuation of application No. 16/152,575, filed on Oct. 5, 2018, now abandoned.
Application No. 16/152,575 is a continuation of application No. 15/306,387, filed on Oct. 24, 2016, now abandoned.
(60) Provisional application No. 61/984,304, filed on Apr. 25, 2014.

(51) Int. Cl. G01N 33/50; G01N 33/574

(52) U.S. Cl. CPC G01N 33/5011; G01N 33/57423; G01N 33/57438

(57) ABSTRACT

The use of genomic tests shows variability between the primary tumor and the metastases in most circumstances referred to as tumor heterogeneity. Since it is unduly invasive and difficult to obtain samples from the primary and metastatic tumors within a patient, a need exists for a method of testing chemotherapeutic effectiveness in a patient that is applicable to both primary tumor and metastases. Provided are methods of using the MiCK assay to determine the most effective drug candidate(s) for an individual patient by testing a single tumor site. In a further embodiment, the kinetic unit (KU) value obtained by analysis of cancer cells from a tumor site in an individual patient in the presence of a drug candidate is within two standard deviations of the KU value obtained by analysis of a different tumor site in the patient in the presence of the same drug candidate.

### CROSS-REFERENCE TO RELATED APPLICATIONS

The present application claims the benefit of U.S. Provisional Application No. 61/984,304, filed Apr. 25, 2014, which is hereby incorporated by reference herein in its entirety.

### BACKGROUND

### 1. Field

The present disclosure relates to use of a spectrophotometric apoptosis (MiCK) assay to determine the efficacy of drug candidate(s) against a primary and/or metastatic tumor by testing the efficacy of drug candidate(s) on a different tumor site in an individual cancer patient.

### 2. Description of Related Art

Cell death may occur in a variety of ways, but most successful anti-cancer drugs tend to cause death of cancer cells by the very specific process of apoptosis. Apoptosis is a mechanism by which a cell disassembles and packages itself for orderly disposal by the body. Apoptosis is commonly used by the body to discard cells when they are no longer needed, are too old, or have become damaged or diseased. In fact, some cells with dangerous mutations that might lead to cancer, and even some early-stage cancerous cells, may undergo apoptosis as a result of natural processes.

During apoptosis, the cell cuts and stores DNA, condenses the nucleus, discards excess water, and undergoes various changes to the cell membrane, such as blebbing, the formation of irregular bulges in the cell membrane. (See FIG. 1) Apoptosis generally occurs after one of several triggers sends a signal to the cell that it should undergo apoptosis. In many cancer cells, this message system does not work correctly because the cell cannot detect the trigger, fails to send a signal properly after the trigger is received, or fails to act on the signal, or the cell may even have combinations of these problems. The overall effect is a resistance to undergoing apoptosis in some cancer cells.

Cancer, as used herein, includes all cancers or malignancies, both hematologic and non-hematologic, as well as myelodysplastic syndromes (MDS). This contemplates the four major categories for all blood/marrow cancers, solid tumors, and effusions: leukemia, lymphomas, epithelial malignancies, and mesenchymal malignancies.

Although many effective cancer drugs can induce cancerous cells to undergo apoptosis despite their resistance to the apoptotic process, no drug works against all types of cancer cells and no test predicts the relative efficacy of these drugs based on kinetic unit measurements of apoptosis. Accordingly, there is a need to detect whether a particular drug candidate can cause apoptosis in various types of cancer cells and also to determine the drug candidate's effectiveness as compared to other drugs or drug candidates, especially with regard to individual patients.

The Microculture Kinetic Assay (MiCK assay), described in U.S. Pat. Nos. 6,077,684 and 6,258,553, is currently used to detect whether cancer cells from a patient undergo apoptosis in response to a particular drug known to be effective against specific cancer types. In the MiCK assay, cancer cells from a patient are placed in a suspension of a given concentration of single cells or small cell clusters and allowed to adjust to conditions in multiple wells of a microtiter plate. Control solutions or solutions with various concentrations of known anti-cancer drugs, typically those drugs recommended for the patient's cancer type, are introduced into the wells with one test sample per well. The optical density of each well is then measured periodically, typically every few minutes, for a period of hours to days. As a cell undergoes apoptosis-related blebbing, its optical density increases in a detectable and specific fashion. If the cell does not undergo apoptosis or dies from other causes, its optical density does not change in this manner. Thus, if a plot of optical density (OD) versus time for a well yields a straight line curve having a positive slope over the time, followed by a plateau and/or a negative slope, then the anti-cancer drug in that well induces apoptosis of the patient's cancer cells and might be a suitable therapy for that patient. OD versus time data may also be used to calculate kinetic units, the units which can be used to measure apoptosis, which similarly correlate with the suitability of a therapy for the patient. One of ordinary skill in the art will be familiar with the aforementioned general description of the MiCK assay. Further, the contents of U.S. Pat. Nos. 6,077,684 and 6,258,553, are herein incorporated by reference in their entirety for all purposes, and provide a more detailed description of the MiCK assay.

Cancer is a multi-clonal disease. There is a general observation that when tumors metastasize there is clonal evolution and the metastases usually have different characteristics from the original or primary tumor. Mutations occur in cancer cells and over time, there is clonal evolution or change in the cells. Gene analyses of metastases reveal differences from the original primary tumor site. These differences represent different clones of the tumor cells. These clones have different characteristics evidenced by: different rates of cell growth of the evolved cells, changed structural appearance when observed under a microscope, changes in hormone receptor status, along with numerous other cell changes. The cellular changes occurring as cancer cells metastasize to multiple locations is evidence of clonal evolution.

The use of genomic tests shows variability between the primary tumor and the metastases in most circumstances. This is referred to as tumor heterogeneity, or multi-clonality. Since it is unduly invasive and often difficult to obtain tumor samples from the primary tumor and each metastatic tumor within an individual patient, it would be advantageous to have a method of testing chemotherapeutic effectiveness in a patient that is applicable to both primary tumor and metastases. Thus, there is a need in the art for a method of identifying the most effective chemotherapy for an individual patient by relying on testing one tumor, not tissue from multiple tumors, within the individual patient.

The solution to this technical problem is provided by the embodiments characterized in the claims.

### BRIEF SUMMARY

The present application relates to methods of using the MiCK assay to determine the most effective drug candidate or combination of drug candidates for an individual patient by testing a single tumor site. The method may include placing a single-cell suspension of viable cancer cells obtained from a tumor site in an individual patient in at least one well of a plate suitable to be read by a spectrophotometer, wherein the cancer cells are in a concentration sufficient to form a monolayer of cells on the bottom of the well, adding at least one drug candidate to the well in an amount sufficient to achieve a target drug candidate concentration, measuring the optical density of the well at a wavelength of approximately 600 nm using a spectrophotometer at selected time intervals for a selected duration of time, determining a kinetic units (KU) value from the optical density and time measurements, and correlating the KU value with an ability of the anti-cancer drug candidate to induce apoptosis in the cancer cells if the KU value is positive, or an inability of the anti-cancer drug candidate to induce apoptosis in the cancer cells if the KU value is not positive.

According to a more specific embodiment, the most effective drug candidate or combination of drug candidates identified for an individual patient by testing a single tumor site is also the most effective drug candidate or combination of drug candidates at other tumor sites in the patient. For example, a method of determining the most effective drug candidate or combination of drug candidates for an individual patient, wherein a sample from the primary tumor site is tested using the MiCK assay, wherein the most effective drug candidates or combination of drug candidates at the primary tumor site is indicative of the most effective drug candidate or combination of drug candidates at a metastatic tumor site is provided herein. Furthermore, a method of determining the most effective drug candidate or combination of drug candidates for an individual patient, wherein a sample from a metastatic tumor site is tested using the MiCK assay, wherein the most effective drug candidate or combination of drug candidates at the metastatic tumor site is indicative of the most effective drug candidate or combination of drug candidates at the primary tumor site is provided herein.

According to additional embodiments, a method of determining the most effective drug candidate or combination of drug candidates for an individual patient, wherein a sample from a primary tumor site or a metastasis of the primary tumor site is tested using the MiCK assay, wherein the most effective drug candidate or combination of drug candidates at the primary tumor site or metastasis thereof is indicative of the most effective drug candidate or combination of drug candidates at all metastasis of said primary tumor is provided herein.

According to a further specific embodiment, the kinetic unit (KU) value obtained by analysis of cancer cells from a tumor site in an individual patient in the presence of a drug candidate or combination of drug candidates is within four standard deviations of the KU value obtained by analysis of a different tumor site in the patient in the presence of the same drug candidate or combination of drug candidates.

According to a further specific embodiment, the kinetic unit (KU) value obtained by analysis of cancer cells from a tumor site in an individual patient in the presence of a drug candidate or combination of drug candidates is within three standard deviations of the KU value obtained by analysis of a different tumor site in the patient in the presence of the same drug candidate or combination of drug candidates.

According to a further specific embodiment, the kinetic unit (KU) value obtained by analysis of cancer cells from a tumor site in an individual patient in the presence of a drug candidate or combination of drug candidates is within two standard deviations of the KU value obtained by analysis of a different tumor site in the patient in the presence of the same drug candidate or combination of drug candidates.

According to a further specific embodiment, the KU value obtained by analysis of cancer cells from a tumor site in an individual patient in the presence of a drug candidate or combination of drug candidates in within one standard deviation of the KU value obtained by analysis of a different tumor site in the patient in the presence of the same drug candidate or combination of drug candidates.

According to additional embodiments, the cancer cells are from a solid tumor. In a preferred embodiment, the solid tumor is a pancreatic tumor or a lung tumor. In a more preferred embodiment, the lung tumor is a non-small cell lung tumor, a small cell lung tumor, or a lung adenocarcinoma.

The following abbreviations and terms are used commonly throughout this Specification:

OD—optical density.
MiCK—microculture kinetic.
KU—kinetic unit.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 shows a time sequences photomicrograph of a cancer cell moving through the stages of apoptosis. The first panel of the left (1) shows the cell prior to apoptosis. The middle panel (2) shows the cell during apoptosis and blebbing is apparent. The last panel on the right (3) shows the cell after apoptosis is complete or nearly complete.

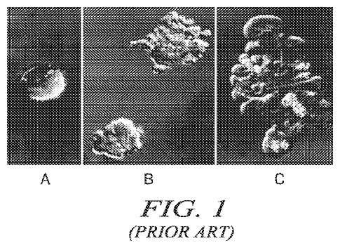

FIG. 2 is a graph showing representative curves for induction of apoptosis, drug resistance, and control cells without drug in a MiCK assay. The curve labeled “B12” shows data representative of cells in which the drug induces apoptosis. The curve labeled “F3” shows data representative of cells that are resistant to the drug. The curve labeled “G5” shows data representative of control cells that did not receive any drug.

FIG. 3 is a graph showing representative data for induction of apoptosis or necrosis in a MiCK assay. The curve labeled “D2” shows data representative of cells in which the drug induces apoptosis. The curve labeled “D7” shows data representative of cells in which the drug induces necrosis or which otherwise undergoes necrosis during the course of the assay.

FIG. 4 is a graph showing representative data for general non-drug-induced cell death in a MiCK assay. The curve labeled “C4” shows data representative of spontaneous cell death during the course of the assay.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

The subject disclosure features, in one aspect, methods of using an assay similar to the microculture kinetic (MiCK) assay, as disclosed in U.S. Pat. Nos. 6,077,684 and 6,258,553, both incorporated by reference herein, to determine the most effective chemotherapeutic drug or combination of chemotherapeutic drugs for an individual patient by testing a single tumor site.

### General MiCK Assay Protocol

According to one specific embodiment, the assay may proceed by selecting an anti-cancer drug candidate and selecting at least one tumor on which to test the drug. Purified cancer cells are obtained from the tumor and the cancer cells may be suspended as a single-cell suspension in culture medium, such as RPMI. As used herein, a “single cell suspension” is a suspension of one or more cells in a liquid in which the cells are separated as individuals or in clumps of 10 cells or fewer. The culture medium may contain other components, such as fetal-bovine serum or components specifically required by the cancer cells. These components may be limited to those necessary to sustain the cells for the duration of the assay, typically at least 24 hours and not longer than 120 hours.

Suspended cells may be tested by placing samples in wells of a spectrophotometric plate. The cells may be suspended at any concentration such that during the spectrophotometric measurements of OD, the beam of the plate reader normally passes through only one cell layer at a time. For most cells, a concentration of between 2×10⁵ and 1×10⁶ cells/mL may be used. Concentration may be increased for small cells and decreased for large cells. To more precisely determine the appropriate cell concentration, the volume of cell suspension to be used in drug candidate test samples may be added to at least one concentration test well of the plate. If the well will be prefilled with additional medium during testing of the drug candidates, then the concentration test well may similarly be prefilled with additional medium. After the concentration test well is filled, the plate may be centrifuged (e.g. for 30 sec to 2 min at 500 RPM) to settle the cells on the bottom of the well. If the cell concentration is appropriate for the assay, the cells should form a monolayer without overlapping. Cell concentration may be adjusted as appropriate until this result is achieved. Multiple concentrations of cells may be tested at one time using different concentration test wells.

According to embodiments where the cells may grow significantly overnight or during another period of time between placement of the cells in the plate and commencement of the drug candidate assay, the cell concentration may be adjusted to initially achieve less than a monolayer to allow for growth such that sufficient cells for a monolayer will be present when the drug candidate assay commences.

The cancer cells may be in an exponential or a non-exponential growth phase. In a specific embodiment, particularly when the cancer cells are from a cancer cell line, they may be in an exponential growth phase.

After the appropriate cell concentration has been determined, the drug-candidate assay may proceed by filling test and control wells in the plate with an appropriate volume of medium and an appropriate number of cells. In other embodiments, the well may be partially pre-filled with medium alone.

After filling, the cells may be allowed to adjust to the plate conditions for a set period of time, such as at least 12 hours, at least 16 hours, at least 24 hours, or 12-16 hours, 12-24 hours, or 16-24 hours. The adjustment period is typically short enough such that the cells do not experience significant growth during the time. The adjustment period may vary depending on the type of cancer cells used in the drug candidate assay. Adjustment may take place under conditions suitable to keep the cells alive and healthy. For example, the plate may be placed in a humidified incubator at 37° C. under 5% CO₂ atmosphere. For some cell types, particularly cell types that do not undergo an adjustment period, the plate may be centrifuged (e.g. for 30 sec to 2 minutes at 500 RPM) to settle the cells on the bottom of the wells.

The drug candidate and any control drugs or other control samples may be added to the wells after the adjustment period. Typically the drug candidate will be added in a small volume of medium or other liquid as compared to the total volume of liquid in the well. For example, the volume of drug added may be less than 10% of the total volume of liquid in the well. Drug candidates may be added in multiple dilutions to allow determination of any concentration effects. Although many drug candidates may be water-soluble, drug candidates that are not readily soluble in water may also be tested. Such candidates may be mixed with any appropriate carrier. Such candidates may preferably be mixed with carriers anticipated for actual clinical use. Viscous drug candidates may require substantial dilution in order to be tested. Drug candidates with a strong color may benefit from monitoring of OD in test wells containing only the drug candidate and subtraction of this OD from measurements for the test sample wells.

After addition of the drug candidate, the cells may be allowed another short period of adjustment, for example of 15 minutes or 30 minutes. The cells may be placed under conditions suitable to keep the cells alive and healthy. For example, the plate may be placed in a humidified incubator at 37° C. under 5% CO₂ atmosphere. After this short adjustment period, a layer of mineral oil may be placed on top of each well to maintain CO₂ in the medium and prevent evaporation.

The plate may then be placed in a spectrophotometer configured to measure the OD at a wavelength of 600 nm for each well at a given time interval for a given total period of time. For example, OD for each well may be measured periodically over a time frame of seconds, minutes, or hours for a period of between 24 and 120 hours. For certain cells, measurements for a period of as little as 12 hours may be sufficient. In specific embodiments, measurements may be taken every 5 to 10 minutes. The spectrophotometer may have an incubated chamber to avoid spontaneous death of the cells.

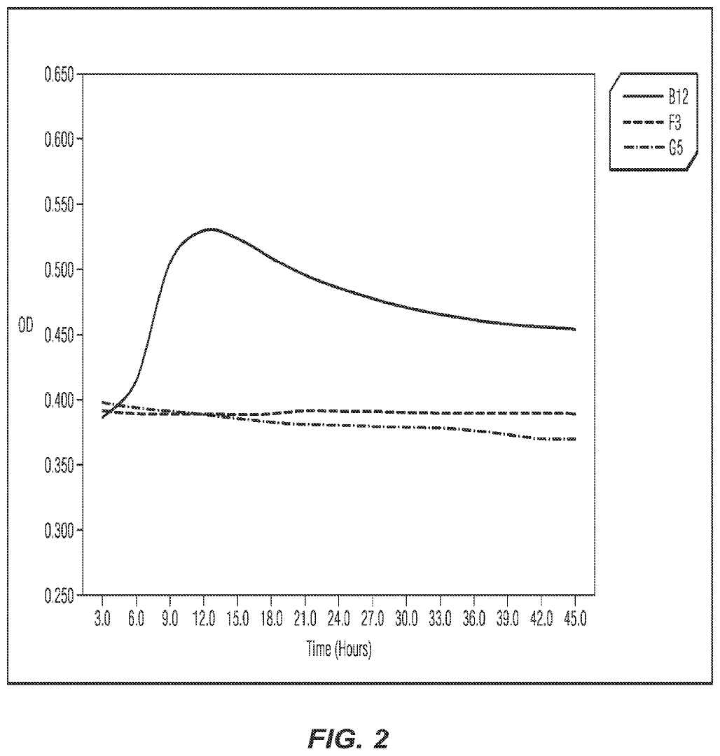

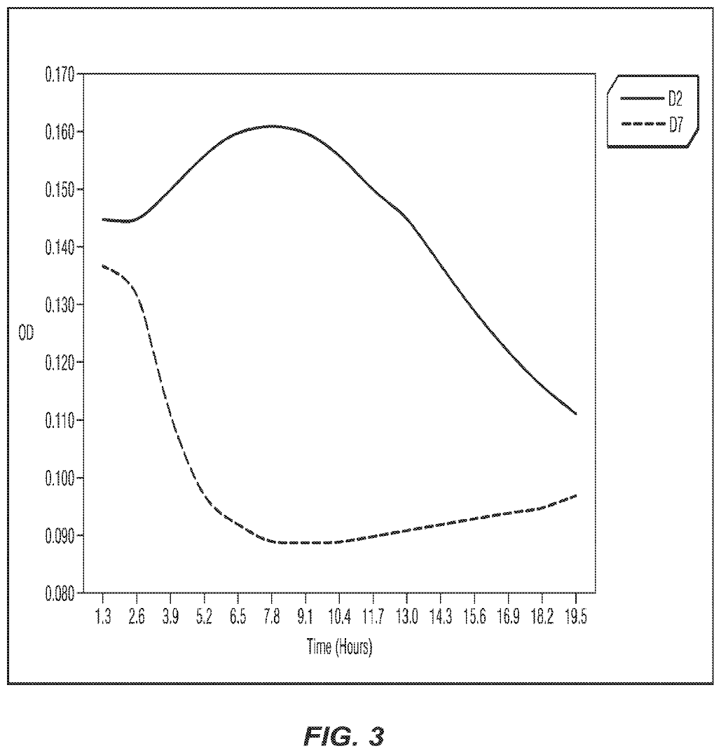

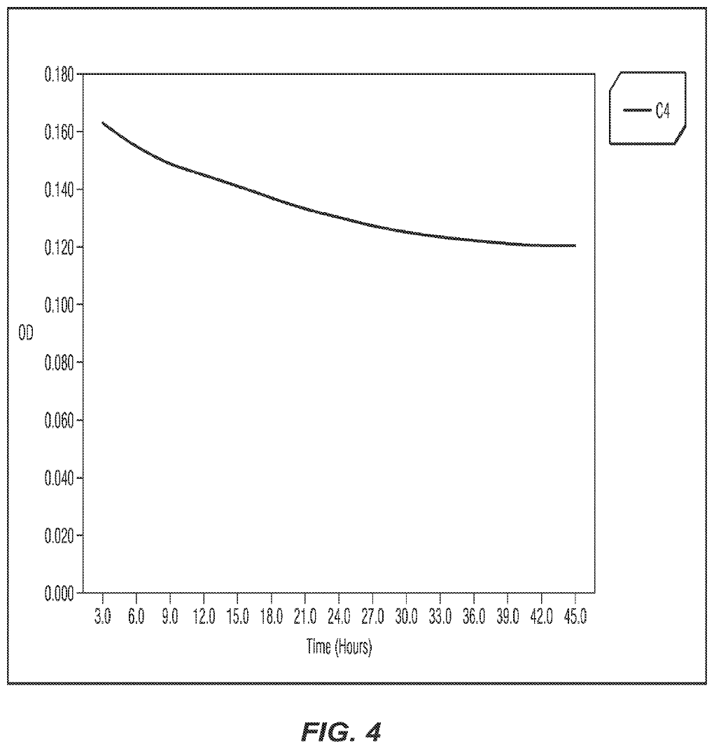

Spectrophotometric data may be converted to kinetic units. Kinetic units are determined by the slope of the curve created when the change in the OD at 600 nm caused by cell blebbing is plotted as a function of time. Specific information regarding the calculation of kinetic units is provided in Kratsov, Vladimir D. et al, Use of the Microculture Kinetic Assay of Apoptosis to Determine Chemosensitivities of Leukemias, Blood 92:968-980 (1998), incorporated by reference herein. Optical density for a given drug candidate at a given concentration may be plotted against time. This plot gives a distinctive increasing curve if the cells are undergoing apoptosis. An example of the curve obtained when cells undergo apoptosis is shown in FIGS. 2 and 3. In comparison, if the drug candidate has no effect on the cells (e.g. they are resistant), then the curve is similar to that obtained for a control sample with no drug or drug candidate (FIG. 2). Cell death due to reasons other than apoptosis can also be determined by the current assay and is useful in eliminating false positives from drug candidate screening. For example, cell necrosis produces a distinctive downward sloping curve easily distinguishable from the apoptosis-related curve as seen in FIG. 3. Further, general cell death also causes a downward curve as seen in FIG. 4.

### Kinetic Units of Apoptosis (KU)

The effectiveness of a drug candidate may be determined by the value of the kinetic units it produces in a modified MiCK assay using a known cell line. Kinetic units may be determined as follows:

Apoptosis(KU)=(Vmax_{Drug Candidate Treated}−Vmax_{Control})×60×X/(OD_{control}−OD_{blank})

The KU is a calculated value for quantifying apoptosis. The optical densities (OD) from each well are plotted against time. The maximum slope of the apoptotic curve (Vmax) is calculated for each plot of drug-treated microculture. It is then compared to the Vmax of a control well without drug (calculated at the same time as the Vmax of the drug exposed cells). For convenience, the Vmax is multiplied by 60 to convert the units from mOD/minute to mOD/hour. The data are normalized with a coefficient (coefficient=X/OD_{control}−OD_{blank}), which is discussed below.

### Coefficient

As stated above, the coefficient is a calculated value for normalizing the amount of cells per well when measuring apoptosis and quantifying said apoptosis in Kinetic Units.

The coefficient is calculated as follows:

Coefficient=X/(OD_{control}−OD_{blank})

X optimal optical density value for the cell type tested (determined empirically).
OD_{control}=average optical density of all the control wells.
OD_{blank}=average optical density of all the blank wells.

A coefficient of 1.000 means that the cell concentration is optimal. A coefficient value below 1.000 means that the cell concentration is higher than the optimal concentration. If the coefficient value is above 1.000, it means that the cell concentration in the well is suboptimal. The acceptable coefficient values for an optimal MiCK assay are between 0.8 and 1.5. If the value is under 0.8, the coefficient will erroneously reduce the value of the calculated KU. If the value is above 1.5, there will not be enough cells per well to detect the signal of apoptosis. The “X” in the formula will vary depending on the cell type. For solid tumor specimens, this value is 0.09. For most of the leukemias, the value is 0.15. For CLL (chronic lymphocytic leukemias) and the lymphomas, the value is 0.21.

This “X” value is adapted to the tumor type and determined empirically. Thus, the coefficient is developed by trial and error, using different concentrations of cells and by checking them under a microscope while looking for complete proper coverage in the well. The proper well is read by a reader and the OD becomes the new X value. Further information regarding this equation may be found in Kravtsov et al. (Blood, 92:968-980), which was previously incorporated herein by reference.

In addition to allowing determinations of whether or not a drug candidate causes apoptosis, kinetic unit values generated using the current assay may be compared to determine if a particular drug candidate may also be performed and may give general indications of appropriate dosage. Occasionally some drugs may perform less well at higher concentrations than lower concentrations in some cancers. Comparison of kinetic unit values for different concentrations of drug candidates may identify drug candidates with a similar profile.

Overall, evaluation of an anti-cancer drug candidate may include any determination of the effects of that drug candidate on apoptosis of a cancer cell. Effects may include, but are not limited to induction of apoptosis, degree of induction of apoptosis as compared to known cancer drugs, degree of induction of apoptosis at different drug candidate concentrations, and failure to induce apoptosis. The anti-cancer drug evaluation assay may also be able to detect non-drug-related or non-apoptotic events in the cancer cells, such as cancer cell growth during the assay or cell necrosis.

Any statistically significant positive kinetic unit value above 1.0 KU may indicate some tendency of a drug candidate to induce apoptosis of a cancer cell. For many clinical purposes, however, drug candidates or concentrations of drugs only able to induce very low levels of apoptosis are not of interest. Accordingly, in certain embodiments of the disclosure, threshold kinetic unit values may be set to distinguish drug candidates able to induce clinically relevant levels of apoptosis in cancer cells. For example, the threshold amount may be 1.5, 2 or 3 kinetic units. The actual threshold selected for a particular drug candidate or concentration of drug candidate may depend on a number of factors. For example, a lower threshold, such as 1.5 or 2, may be acceptable for a drug candidate able to induce apoptosis in cancer types that do not respond to other drugs or respond only to drugs with significant negative side effects. A lower threshold may also be acceptable for drug candidates that exhibit decreased efficacy at higher concentrations or which themselves are likely to have significant negative side effects. A higher threshold, such as 3, may be acceptable for drug candidates able to induce apoptosis in cancer types for which there are already suitable treatments.

In another embodiment, the following threshold ranges can be utilized:

0-1 KU=non-sensitive;
1-2 KU=low sensitivity;
2-3 KU=low/moderate sensitivity;
3-5 KU=moderate sensitivity; and
>5 KU=sensitive.

Preferably, the following threshold ranges can be utilized:

0-1 KU=non-sensitive;
1-2.6 KU=low sensitivity;
2.6-4.2 KU=low/moderate sensitivity;
4.2-5.8 KU=moderate sensitivity; and
>5.8 KU=very sensitive.

Preferably, the KU value is 7, more preferably the KU value is 8, even more preferably the KU value is 9, and most preferably the KU value is 10.

These ranges were established based on a statistical analysis of cancer cells. The ranges establish a baseline for relative comparison of chemotherapeutic drugs being tested on a specific cell type. Test outcomes may be affected by extenuating factors such as: time elapsed from obtaining sample to testing; quantity of viable cells available to test; microbial contamination of specimen; quality or viability of cells being tested; cell type; and recent treatment such as chemotherapy or radiation therapy.

These factors suggest some elasticity in the predictive values of the kinetic response reported. Clinical sensitivity to chemotherapy drugs is not completely limited to outcomes as forecast in the above ranges. The KU measurement of drug-induced apoptosis in the assay may be used by physicians to develop an individual patient treatment regimen along with other important factors such as patient history, prior treatment results, overall patient health, patient comorbidities, patient preferences, as well as other clinical factors.

Therefore, the particular ranges of KU value utilized will be dependent upon context. That is, depending upon the particular type of tumor cell being tested, the particular drug being utilized, and the particular patient or patient population under analysis. The KU value therefore represents a dependable and flexible analytical variable that can be tailored by the practitioner of the disclosed methods to create a suitable metric by which to evaluate a given drug's effect.

### Drug Candidates

According to a specific embodiment, the anti-cancer drug candidates may be any chemical, chemicals, compound, compounds, composition, or compositions to be evaluated for the ability to induce apoptosis in cancer cells. These candidates may include various chemical or biological entities such as chemotherapeutics, other small molecules, protein or peptide-based drug candidates, including antibodies or antibody fragments linked to a chemotherapeutic molecule, nucleic acid-based therapies, other biologics, nanoparticle-based candidates, and the like. Drug candidates may be in the same chemical families as existing drugs, or they may be new chemical or biological entities.

Drug candidates are not confined to single chemical, biological or other entities. They may include combinations of different chemical or biological entities, for example, proposed combination therapies. Further, although many examples herein relate to an assay in which a single drug candidate is applied, assays may also be conducted for multiple drug candidates in combination. It is also important to understand that embodiments of the invention may utilize the metabolites of the various drug candidates in a method as described.

Embodiments of the invention are able to test all manner of anti-cancer drug candidates. For example, the following anti-cancer drug candidates can be tested by the disclosed methods: Abraxane, Afatinib, Alimta, Amsacrine, Asparaginase, BCNU, Bendamustine, Bleomycin, Bosutinib, Caelyx (Doxil), Carboplatin, Carmustine, CCNU, Chlorambucil, Cisplatin, Cladribine, Clofarabine, Cytarabine, Cytoxan (4HC), Dacarbazine, Dactinomycin, Dasatinib, Daunorubicin, Decitabine, Dexamethasone, Doxorubicin, Epirubicin, Estramustine, Etoposide, Fludarabine, 5-Fluorouracil, Gemcitabine, Gleevec (imatinib), Hexamethylmelamine, Hydroxyurea, Idarubicin, Ifosfamide (4HI), Interferon-2a, Irinotecan, Ixabepilone, Melphalan, Mercaptopurine, Methotrexate, Mitomycin, Mitoxantrone, Nilotinib, Nitrogen Mustard, Oxaliplatin, Pentostatin, Sorafenib, Streptozocin, Sunitinib, Tarceva, Taxol, Taxotere, Temozolomide, Temsirolimus, Teniposide, Thalidomide, Thioguanine, Topotecan, Tretinoin, Velcade, Vidaza, Vinblastine, Vincristine, Vinorelbine, Vorinostat, Xeloda (5DFUR), Everolimus, Lapatinib, Lanalidomide, Rapamycin, and Votrient (Paxopanib).

However, many other anti-cancer drug candidates, including but not limited to other nonchemotherapy drugs and/or chemicals which can produce apoptosis or which are examined for their ability to produce apoptosis, are also able to be tested by the disclosed methods.

Further still, the methods of the present invention are not strictly applicable to anti-cancer drug candidates, but rather embodiments of the disclosed methods can be utilized to test any number of potential drug candidates for a whole host of diseases.

More than one drug candidate, concentration of drug candidate, or combination of drugs or drug candidates may be evaluated in a single assay using a single plate. Different test samples may be placed in different wells. The concentration of the drug candidate tested may be, in particular embodiments, any concentration in the range from 0.1 to 10,000 μM, or any concentration in the range from 0.01 to 10,000 μM, or any concentration in the range from 0.001 to 100,000 μM, for example. The concentration tested may vary by drug type, and the aforementioned example concentrations are not to be considered as limiting, for the skilled artisan will understand how to construct the appropriate concentration for utilization with the taught methods and assays, depending upon the particular anti-cancer drug tested.

### Plate and Spectrophotometer Systems

In specific embodiments, the plate and spectrophotometer may be selected such that the spectrophotometer may read the plate. For example, when using older spectrophotometers, one may use plates with larger wells because the equipment is unable to read smaller-well plates. Newer spectrophotometers may be able to read a plate with smaller wells. In one embodiment, the diameter of the bottom of each well is no smaller than the diameter of the light beam of the spectrophotometer. In a more specific embodiment, the diameter of the bottom of each well is no more than twice the diameter of the light beam of the spectrophotometer. This helps ensure that the OD at the measured wavelength, 600 nm for example, of a representative portion of the cells in each well is accurately read. The spectrophotometer may make measurements at wavelengths of than 600 nm. For example, the wavelength may be +/−5 or +/−10. However, other wavelengths may be selected so as to be able to distinguish blebbing.

Spectrophotometers may include one or more computers or programs to operate the equipment or to record the results. In one embodiment, the spectrophotometer may be functionally connected to one or more computers able to control the measurement process, record its results, and display or transmit graphs plotting the optical densities as a function of time for each well.

Plates designed for tissue culture may be used, or other plates may be sterilized and treated to make them compatible with tissue culture. Plates that allow cells to congregate in areas not accessible to the spectrophotometer, such as in corners, may work less well than plates that avoid such congregation. Alternatively, more cancer cells may be added to these plates to ensure the presence of a monolayer accessible to the spectrophotometer during the assay. Plates with narrow bottoms, such as the Corning Costar® half area 96-well plate, may also assist in encouraging formation of a monolayer at the bottom of the well without requiring inconveniently low sample volumes. Other plates, such as other 96-well plates of smaller well plates, such as 384-well plates, may also be used.

### Modified MiCK Assay Protocol

A modified MiCK assay protocol has recently been developed as described in International Patent Application Publication WO 2013/172955, incorporated by reference herein. This modified assay protocol is particularly suitable for the study of solid tumors. Specifically, adherence of cancer cells to the well bottom is required for testing cancers and sarcomas that are not of blood or bone marrow origin because these cells require a permanent close contact with each other due to the nature of solid tumors. Accordingly, in a preferred embodiment, a method of determining the most effective drug candidate or combination of drug candidates for an individual patient, wherein a sample from a primary or metastatic site is tested using a MiCK assay that has been modified as described in this section.

In particular, the MiCK assay may be modified, for example, by:

a. overnight incubation for solid tumor sample specimens;
b. use of low volume wells since solid tumors usually give fewer cells than blood samples;
c. adjusting cell concentration via visual interpretation;
d. allowing cells to adhere to the bottom of the wells and spread/stretch overnight;
e. utilization of a special incubation chamber to diffuse heat evenly;
f. avoiding the edges of the plates when one loads the cells into the wells;
g. utilization of an automated pipettor to plate the cells, media (e.g., RPMI+10% Fetal Bovine Serum+Penstrep) and drugs; and
h. utilization of proprietary code created to translate template in a format that a robot can understand.

In addition, cell isolation and plating can be modified as follows:

A cell count from a pure cell suspension is used to adjust the cell concentration to 1×10⁶ cells/mL. A test well is plated to observe the cell distribution. If the cells are not in good shape, more cells are added to each well. If the test well seems adequate (monolayer of uniformly distributed cells that covers the bottom of the well), one proceeds to the next step (plating). If the test well is not adequate, adjustment of the cell concentration (e.g., diluting the cells or concentrating the cells) and retesting a new well is repeated until the cell distribution in the well is satisfactory.

After the aforementioned steps, the stock solution is ready to be plated into additional wells in the plate until the cells are depleted. Using the selected cell concentration, the cell suspension is distributed in the plate into as many wells as possible, retaining enough cells to do at least 1 cytospin and immunocytochemistry (ICC) if possible.

An automated pipettor is used to distribute the cells while avoiding the edge wells of the plates; the edge wells are filled with media. Optimal liquid dispensing parameters were developed to prevent air bubble formation while the drugs are added to the wells. This feature is important as it eliminates the formation of bubbles in the media during the assay which artificially elevate the slope values which leads to markedly elevated KU values.

Once the plate has undergone the aforementioned steps, it is ready for overnight incubation (approximately 15 hour); allowing time for the cells to adhere to the bottom of the wells as well as to stabilize metabolically.

After the incubation plate is removed from the incubator, the cell distribution and viability are evaluated from an observation of the plate with an inverted microscope. A photomicrograph of a representative well is taken and the plate is then ready for addition of the drugs (e.g., possible anti-cancer agents) by the automated pipettor. Drugs are selected by the treating oncologist (for example), and NCCN panels, then off panel drugs (off label).

In some embodiments, each well of the plate comprises a different anti-cancer drug candidate. Further, the method also contemplates embodiments in which a different concentration of the anti-cancer drug candidate is contained in each well. Therefore, the present disclosure may relate to high-throughput assays by which multiple potential drug candidates at multiple potential concentration strengths may be simultaneously tested.

The potential anti-cancer drug candidate concentration which may be loaded into each well of the assay will vary depending upon the manufacturer's recommended dosage and the corresponding dilutions required to achieve the concentration in the well that would correspond to this dosage. For example, the target drug concentration in each well is determined by molarity and can range from 0.01 to 10,000 μM, or 0.001 to 100,000 μM, or 0.1 to 10,000 μM for example, but could also deviate from these disclosed example ranges or comprise any integer contained within these ranges. One skilled in the art will understand how to achieve a target drug concentration by utilizing the manufacturer's recommended blood level concentrations, which may vary plus or minus one serial dilution if enough specimen cells are present.

Once the drug candidates are added to the wells, an incubation of 30 minutes at 37° C. and 5% CO₂ is done to allow for pH equilibration; oil is added to every well to prevent air exchange and evaporation; the plate is placed in a reader and the assay is started; the assay automatically terminates after 576 reads (48 hours, 5 min intervals). These settings can be adjusted as needed and the assay can be manually terminated if all the reactions are deemed to have been completed prior to the 48 hours.

A trained observer may assess cytologic characteristics of cells at all stages of purification. A trained observer may also analyze ranking of drugs; analyze best drugs or combinations; and analyze most active drug candidates (may also include analyzing drug metabolites) and other developed drugs or agents.

### Cancer Cells

The methods described herein may be used to determine the most effective drug candidate for the treatment of an individual cancer patient. In a preferred embodiment, the cancer is a sarcoma, lymphoma, carcinoma, germ cell tumor and/or blastoma. In a further preferred embodiment, the cancer is a lung cancer, a breast cancer, a liver cancer, a colon cancer, a pancreatic cancer, a colorectal cancer, an ovarian cancer, a uterine cancer, a testicular cancer, a prostate cancer, a central nervous system cancer, a cancer of the head and neck, an endothelioma, an osteoblastoma, an osteoclastoma, Ewing's sarcoma and/or Kaposi's sarcoma.

In a specific embodiment, the cancer may be a solid tumor such as, but not limited to, follicular lymphomas, carcinomas with p53 mutations, and hormone-dependent tumors, including, but not limited to, colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer.

In a further preferred embodiment, the cancer may be a solid tumor including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Embodiments of the present invention may be utilized to test a wide variety of malignancies. For example, the present disclosure may be used to test the following carcinomas:

Ovarian carcinoma (serous cystadenocarcinoma, mucinous cystadenocarcinoma, endometroid carcinoma), Ovarian granulosa cell tumor, Fallopian tube adenocarcinoma, Peritoneal carcinoma, Uterine (endometrial) adenocarcinoma, sarcomatoid carcinoma, Cervical squamous cell carcinoma, Endocervical adenocarcinoma, Vulvar carcinoma, Breast carcinoma, primary and metastatic (ductal carcinoma, mucinous carcinoma, lobular carcinoma, malignant phyllodes tumor), Head and neck carcinoma, Oral cavity carcinoma including tongue, primary and metastatic, Esophageal carcinoma, squamous cell carcinoma and adenocarcinoma, Gastric adenocarcinoma, malignant lymphoma, GIST, Primary small bowel carcinoma, Colonic adenocarcinoma, primary and metastatic (adenocarcinoma, mucinous carcinoma, large cell neuroendocrine carcinoma, colloid carcinoma), Appendiceal adenocarcinoma, Colorectal carcinoma, Rectal carcinoma, Anal carcinoma (squamous, basaloid), Carcinoid tumors, primary and metastatic (appendix, small bowel, colon), Pancreatic carcinoma, Liver carcinoma (hepatocellular carcinoma, cholangiocarcinoma), Metastatic carcinoma to the liver, Lung cancer, primary and metastatic (squamous cell, adenocarcinoma, adenosquamous carcinoma, giant cell carcinoma, non-small cell carcinoma, NSCLC, small cell carcinoma, neuroendocrine carcinoma, large cell carcinoma, bronchoalveolar carcinoma), Renal cell (kidney) carcinoma, primary and metastatic, Urinary bladder carcinoma, primary and metastatic, Prostatic adenocarcinoma, primary and metastatic, Brain tumors, primary and metastatic (glioblastoma, multiforme, cerebral neuroectodermal malignant tumor, neuroectodermal tumor, oligodendroglioma, malignant astrocytoma), Skin tumors (malignant melanoma, sebaceous cell carcinoma), Thyroid carcinoma (papillary and follicular), Thymic carcinoma, Shenoidal carcinoma, Carcinoma of unknown origin, primary and metastatic, Neuroendocrine carcinoma, Testicular malignancies (seminoma, embryonal carcinoma, malignant mixed tumors), and others.

The present disclosure may be used to test the following malignant lymphomas, for example: Large cell malignant lymphoma, Small cell lymphoma, Mixed large and small cell lymphoma, Malt lymphoma, Non Hodgkins malignant lymphoma, T cell malignant lymphoma, chronic myelogenous (or myeloid) leukemia (CML), myeloma, other leukemias, mesothelioma, mantle cell lymphomas, marginal cell lymphomas, lymphomas not otherwise specified as to type, and others.

Further still, the present disclosure may be used to test the following sarcomas, for example: Leimyosarcoma (uterine sarcoma), GIST-gastrointestinal stromal tumor, primary and metastatic (stomach, small bowel, colon), Liposarcoma, Myxoid sarcoma, Chondrosarcoma, Ostersarcoma, Ewings sarcoma/PNET, Neuroblastoma, Malignant peripheral nerve sheath tumor, Spindle cell carcinoma, Embryonal rhabdomyosarcoma, Mesothelioma, and others.

### Cancer Cell Preparation

In a further embodiment, cancer cells from solid tumor sites may be prepared by a method comprising:

a. obtaining a tumor specimen;
b. mincing, digesting, and filtering the specimen;
c. optionally removing non-viable cells by density gradient centrifugation;
d. incubating the cell suspension to remove macrophages by adherence;
e. performing positive, negative, and/or depletion isolation to isolate the cells of interest;
f. removing any remaining macrophages, if necessary, using CD14 antibody conjugated magnetic beads; and
g. plating and testing the final cell suspension as described herein.

### EXAMPLES

The following Examples describe exemplary embodiments of the invention. These Examples should not be interpreted to encompass the entire breadth of the invention.

### Example 1—Intratumoral Homogeneity of Drug-Induced Apoptosis (MiCK) Assays in Non-Small Cell Lung Cancer

### Background:

Blinded clinical trials have demonstrated higher response rates and longer survival in groups of patients with acute myelocytic leukemia and epithelial ovarian cancer who have been treated with drugs that show high apoptosis as determined by the MiCK assay (Bosserman et al., Cancer Res 2012; 72:3901-3905). Thus, the MiCK assay provides great potential to guide treatment decisions in individual patients. However, many analyses indicate significant heterogeneity between primary tumor and metastatic sites in individual patients, indicating that multiple tumor sites in a patient must be analyzed to provide accurate therapeutic recommendations. This is not ideal since many times it is difficult or even impossible to obtain samples from multiple tumors in an individual patient. This study aimed to detect drug-induced apoptosis using the MiCK® assay in multiple tumor sites in individual patients.

### Methods:

Summary: Patients with non-small cell lung cancer (NSCLC) or mesothelioma had tumors from different sites sent independently for drug-induced apoptosis analysis as described (Salom et al., J Trans Med 2012; 10:162). Purified tumor cells were cultured for 48 hours with individual drugs, and drug-induced apoptosis was measured using the MiCK assay as described herein. Data were obtained optically using Mie light-scattering. Results from paired tumor sites in individual patients were compared and analyzed statistically. Active apoptosis (sensitivity) was defined as >=1.0 kinetic units (KU), and no apoptosis (resistance) was <1.0 KU, and 2 standard deviations (SD) in the assay were 1.14 KU.

Patients: Five patients with non-small cell lung cancer (NSCLC) including adenocarcinoma and 1 patient with lung cancer probable mesothelioma were included in this study. Two tumor samples from different tumor sites were collected from each patient. Both samples from each patient were collected on the same day. Patient A was diagnosed with lung cancer probable mesothelioma; sample 1 was collected from solid tumor and sample 2 was collected from pleural effusion. Patient B was diagnosed with NSCLC; samples 1 and 2 were collected from different sites of the same solid tumor. Patient C was diagnosed with multi-focal, recurrent NSCLC adenocarcinoma; samples 1 and 2 were collected from separate sections of the right lung. Patient D was diagnosed with multi-focal, recurrent NSCLC adenocarcinoma; sample 1 was collected from the right lung and sample 2 was collected from lymph node. Patient E was diagnosed with multi-focal NSCLC adenocarcinoma; samples 1 and 2 were collected from separate sections of the right lung. Ages of the patients ranged from 47-83 years old with an average age of 72.2 years. Three patients were male, one was female, and one patient is unknown.

### Solid Tumor Cell Specific Isolation Protocol:

Within 24 to 48 hours of collection, the specimen was treated as follows in order to purify and isolate cells from solid tumors:

Take the specimen out of the transport tube.

Put in a petri dish in 13 mL of PBS+high concentration of antibiotics (200 units/mL Penicillin+200 μg/mL streptomycin) and take measurements and picture of the specimen. The PBS+antibiotic solution is made from solutions mixed together in the lab using proprietary protocols.

Wash 3 times in petri dishes (3 different petri dishes) with 13 mL of PBS+high concentration of antibiotics (200 units/mL Penicillin+200 μg/mL streptomycin).

If contamination is suspected, incubate 20 min in a tube with PBS+high concentration of antibiotics.

Transfer the specimen into another petri dish with 1 to 3 mL (depending on specimen size) of RPMI 50% Fetal Bovine Serum (FBS) for mincing.

Next, the specimen was minced, and incubated with a digestion enzyme (enzyme can vary with tissue being used) and 0.08% DNase for 1-2 hours at 37° C. If contaminating non-tumor tissue is identified in the specimen, remove these parts with scalpels. Mince in 1 mm pieces with scalpels size 10 or 21. Collect the pieces with forceps, put in a 15 mL tube+10-12 mL of enzyme (the enzyme depends on the tumor type; see Table 1), incubate 45-60 min in the incubator at 37° C. on a “rotator”. Wash the petri dish used for mincing with RPMI (4-5 mL), 2-3 times. Put the washes in a 15 mL tube, let settle 2-3 min. Remove the supernatant and put in a new 15 mL tube, check the viability of cells with the hemacytometer and trypan blue dye (this gives an early indication on how difficult and/or easy the processing should be). Put the pellet in a 15 mL tube with the enzyme and incubate at 37° C. on the rotator for 45-60 min. After the incubation, collect the supernatant and put back the remaining pieces in fresh enzyme at 37° C. for another 45-60 min.

Next, the specimen was filtered through a 100 micrometer cell strainer. Depending on tumor type and amount of “non-cancer cell tissue” remaining, one could also use 40 and 70 μM strainer or filcon. If the supernatant is viscous or if it contains a lot of debris, it will block the cell strainer. In that case, one may make the determination to perform a “pre-filtration” using sterile gauze over a 50 mL tube. Then proceed with the cell strainer filtration process referenced above.

Centrifuge the filtered cell suspension at 1500 RPM for 5 min.

Discard the supernatant. To the pellet, add 5 mL of red blood cell lysis solution (standard NH₄Cl containing lysis solution: NH₄Cl 0.15M+KHCO₃ 10 mM+EDTA-4Na 0.1 mM, pH 7.2), incubate 2-3 min and add 5 mL of RPMI 10% FBS.

Centrifuge 5 min at 1500 RPM. Resuspend the pellet in RPMI 10% FBS (1-10 mL, depending on the pellet size).

Collect the second fraction in the enzyme and repeat the steps above.

Check the viability of all fractions and pool. Do a cytospin stained with Wright Giemsa to verify the cell content of the population. NOTE: this is done numerous times during the process of purification.

When necessary, non-viable cells were removed by density gradient centrifugation. Density gradient centrifugation (optiprep): first layer=2 mL cells+4.45 mL optiprep 40% in RPMI, second layer=optiprep 22.5% in RPMI, third layer=0.5 mL of RPMI. Centrifuge at 2000 RPM for 20 min.

Collect the viable cell layer, add 10 mL of RPMI 10% FBS, centrifuge at 1500 RPM for 5 min.

Resuspend the pellet in RPMI 10% FBS (volume depends on the pellet size and on the next step required).

If mucin is present in the specimen: resuspend the pellet in 10 mL of PBS+20 mM DTT and incubate at 4° C. for 30 min to disintegrate the mucin. Wash with RPMI at 1500 RPM for 5 min. Resuspend the pellet in RPMI 10% FBS.

If the specimen is highly necrotic with the presence of debris: Percoll 20% in HBSS, centrifuge at 800×g for 10 min.

The cell suspension was then incubated for 20 min at 37° C. in a tissue culture flask to remove macrophages by adherence. The size and quantity of the flask and the volume used depends on the amount of cells. Examples: 1-5×10⁶ cells=25 cm₂ flasks, 3-4 mL each; 1×10⁷ cells=75 cm₂ flasks, 8 mL each; 1×10⁸ cells=175 cm₂ flasks, 20 mL each. After incubation, collect the cell suspension, wash the flask 3 times with RPMT 10% FBS, pool all the washing fractions, and centrifuge at 1500 RPM for 5 min.

For epithelial tumors, lymphocytes were removed by 30 minutes incubation with CD2 antibody conjugated magnetic beads for T lymphocytes and CD19 antibody conjugated magnetic beads for B lymphocytes. Beads to use: T lymphocytes=CD2; B lymphocytes=CD19; neutrophils=CD15; monocytes/macrophages=CD14, all leukocytes=CD45 (use CD45 if there are no clumps). Macrophages are usually removed by adherence, not with the beads. The reason is that if clumps of tumor cells are present, they can also contain macrophages. If beads are used to remove the macrophages, tumor cells could be removed at the same time. Resuspend the pellet in a small volume of PBS 2% FBS (0.2 to 2 mL). Wash the bead suspension 3 times with the PBS 2% FBS. Add the beads to the cell suspension and incubate for 30 min at room temperature on the rotator. Put the tube on the magnet, wait for 1 min. Collect the cell suspension, put in a 15 mL tube with 5 mL of RPMI 10% FBS. Put the tube of the cell suspension on a magnet again to remove remaining beads, collect the cell suspension and put into a new 15 mL tube. Centrifuge at 1500 RPM for 5 min. Resuspend in RPMI 10% FBS, the volume depends on the pellet size. Do a cell count and determine viability, do a cytospin to determine cell content.

Remaining macrophages were removed, if necessary, using CD14 antibody conjugated magnetic beads. This step would be done at the same time that the other beads are being processed as outlined above. Look at the cell viability. An additional step may be required in the viability is less than 80-85%. If that is the case, repeat the density gradient centrifugation (optiprep) as described above. This will remove the dead cells.

The final cell suspension was plated into a 96-well half-area plate, or a 384-well plate with 62.5 μL aliquot per well, or a 384-well plate with a 20 μL aliquot per well, as indicated in Table 2. Adjust the cell concentration to 1×10⁶ cells per mL. Do a test well. For Corning 384=15 μL of RPMI 10% FBS+45 μL of cell suspension, then centrifuge at 500 RPM for 1 min. For Greiner=2.5 μL of RPMI 10% FBS+15 μL of cell suspension, then centrifuge at 500 RPM for 30 sec. Look at the well under the inverted microscope. The cells should touch each other but not be overlapping. Adjust the cell concentration as needed by concentrating (centrifuge and remove medium) or diluting (adding medium). Repeat until optimal cell concentration is found. Plate the cells into the wells.

The plate was incubated overnight at 37° C. with 5% carbon dioxide humidified atmosphere. 5×10⁴ to 1.5×10⁵ cells were seeded per well depending on the cell volume to give adequate well-bottom coverage. The plate was incubated inside a humidity chamber where heat distribution and humidity are optimized to reduce the “edge effect” (bad cell distribution in the well).

RPMI-1640 medium without phenol red was used for all cultures.

### Effusion Specific Isolation Protocol:

Within 24 to 48 hours of collection, the specimen was treated as follows:

Transfer the specimen into 50 mL tubes and take also a 10 mL aliquot in a 15 mL tube (centrifuge the aliquot at 2000 RPM for 5 min, do a cell count and prepare a cytospin to give an idea of the cell content and count of the specimen).

Centrifuge the tubes at 2000 RPM for 15 min.

Remove the supernatant but leave ˜5 mL per tube. Combine all the tubes and dilute 1:1 with PBS in as many 50 mL tubes as needed. Centrifuge 10 min at 2000 RPM.

Do RBC lysis for 2-3 min. The volume depends on the pellet size. Add an equal volume of RPMI 10% FBS.

Centrifuge 1500 RPM for 5 min.

Resuspend the pellet in RPMI 10% FBS, the volume depends on the pellet size.

Do a cell count and determine viability.

Viability is critical to the entire process. It must be determined if the viability is less than ˜70%. If so, do an optiprep centrifugation.

If the viability meets the acceptable standard, and if the major contaminating cells are macrophages, these cells are removed via adherence.

If there is a high contamination from a major cell type and the total cell count is high (5×10⁷ cells or more), do a first purification step with CD45 beads (1 bead per cell). Then repeat the beads a second time and a third time if necessary.

Do a cell count and determine viability.

Repeat optiprep if necessary as recommended by Pathologist.

Coefficient Adjustment—Adjust the coefficient as for the solid tumor specimen based on recommendation of Pathologist.

When the optimal cell concentration is reached, put the cells in the plate and incubate overnight in the incubating chamber of the incubator (37° C.).

MiCK Assay protocol:

The MiCK assay procedure was adapted from the method described in U.S. Pat. Nos. 6,077,684 and 6,258,553, both patents incorporated herein by reference in their entirety. Also, the MiCK assays described in: Kravtsov V. et al, “Use of the Microculture Kinetic Assay of apoptosis to determine chemosensitivities of leukemias.” Blood 1998; 92:9680980, is incorporated herein by reference in its entirety for all purposes.

After overnight incubation, chemotherapy drugs were added to the wells of the 96-well plate in 5 μL aliquots or to the wells of a 384-well plate in 2.5 μL aliquots using an automated pipettor. The number of drugs or drug combinations and the number of concentrations tested depended on the number of viable malignant cells that were isolated from the tumor specimen. The drug concentrations, determined by molarity, were those indicated by the manufacturer as the desired blood level concentration plus or minus one serial dilution if enough cells were available.

Following drug addition, the plate was incubated for 30 min. at 37° C. in a 5% carbon dioxide humidified atmosphere incubator. Each well was then overlayed with sterile mineral oil, and the plate was placed into the incubator chamber of a microplate spectrophotometric reader. The optical density at 600 nanometers was read and recorded every 5 minutes over a period of 48 hours. Optical density increases, which correlates with apoptosis, were converted to kinetic units (KU) of apoptosis by a proprietary software ProApo with a formula described in the Kravtsov reference incorporated by reference (i.e. Kravtsov V. et al. “Use of the Microculture Kinetic Assay of apoptosis to determine chemosensitivities of leukemias.” Blood 1998; 92:968-980). Active apoptosis was indicated as >1.0 KU. A drug producing ≤1 KU was described as inactive, or that the tumor was resistant to that drug based on previous laboratory correlations of KU with other markers of drug-induced cytotoxicity (growth in culture, thymidine uptake). Two standard deviations (SD) in the assay were 1.14 KU.

### Results:

Ten paired specimens were obtained from 5 NSCLC patients and 1 mesothelioma patient. The mean number of individual drug apoptosis tests analyzed per patient was 20.6 (range 3 to 39). Data obtained using the MiCK assay for these ten paired specimens are summarized in Table 3. There was concordance (within 2 SD) of drug-induced apoptosis in 97/103 assays (94%; 95% confidence interval [CT] 84%-100%). Paired specimens analyzed for sensitivity or resistance (< or >1.0 KU) showed concordance (within 2 SD (1.14 KU)) in 95% of assays (CT 85%-100%).

The rate of best drug(s) from specimen A (±2 SD 1.14 KU) also being a best drug from specimen B in the same patient was 8/13, 4/10, 24/24, 4/9, and 1/1. There was a concordance (within 2 SD) of 72% (95% CI 36%-100%). Conversely, the rate of best drug(s) from specimen B (±2 SD 1.14 KU) also being a best drug from specimen A in the same patient was 8/25, 4/5, 24/30, 4/9, and 1/2. There was a concordance (within 2 SD) of 58% (95% CI 27%-89%).

### Conclusions:

This study indicates that drug-induced apoptosis measured by the MiCK assay in different tumor sites is homogeneous defined as within two (2) standard deviations (SD). That is, if tumor cells are biopsied from one area, the same test outcome is observed if cells are biopsied from another location in the same person (within the definition of 2 SD). An additional benefit from this discovery is that tumor cells from multiple organ sites do not have to be harvested and tested.

Using Kinetic Unit (KU) values to measure the effectiveness of a given chemotherapy drug to produce drug-induced apoptosis, the uniqueness of this finding is that 95% of the time there is correlation in test outcomes within two (2) standard deviations (SD). Thus, evidence concludes that the response to therapy is generally homogeneous within this defined range. Homogeneity in the tumor cells from different organ sites is demonstrated by the ability of the same chemotherapy drug to induce apoptosis in the tumor cells in different locations. In addition, when the drug-induced apoptosis is measured and the response of the tumor cell to each individual drug is characterized as sensitive (greater than or equal to 1 KU) or resistant (less than 1 KU), there is agreement in sensitivity or resistance in 94% of assays.

The strength of this finding is significant when the use of the standard criterion is applied: the test result from one organ site is within 2 SD of the second test result. The uniqueness of this discovery is that 94% of the time this is true using the test results from the MiCK® assay.

This study and above data is a discovery of significant finding. Heretofore, the general assumption of primary tumor cells and metastatic tumor cells (with their origin being the primary tumor site) involved their heterogeneity, or unique differences in sensitivity to chemotherapy. The finding that 94% of the cancer cells tested are homogeneous in apoptotic response to chemotherapy drugs is a remarkable discovery. This strongly suggests that there is homogeneity in drug-induced apoptosis as determined using in the MiCK® assay in lung cancer. In NSCLC, the drug-induced apoptosis determined using the MiCK® assay identifies chemotherapy regimens (combinations) which produce highest apoptosis.

### Example 2—Intratumoral Homogeneity of Drug-Induced Apoptosis (MiCK) Assays in Multiple Solid Tumor Cancers

### Background:

Given the homogeneity of drug-induced apoptosis as determined by the MiCK assay in non-small cell lung cancer described in Example 1, we sought to determine whether the drug-induced apoptosis detected using the MiCK® assay also indicated intratumoral heterogeneity in other types of solid tumors.

### Methods:

Patients with pancreatic cancer, lung adenocarcinoma, or small cell lung carcinoma had tumors from different sites sent independently for drug-induced apoptosis analysis as described (Salom et al., J Trans Med 2012; 10:162). Purified tumor cells were cultured for 48 hours with individual drugs, and drug-induced apoptosis was measured using the MiCK assay as described in Example 1. Data were obtained optically using Mie light-scattering. Results from paired tumor sites in individual patients were compared.

Patients: Three patients were included in this study. Two tumor samples from different tumor sites were collected from each patient. Both samples from each patient were collected on the same day. Patient A was diagnosed with pancreatic islet cell cancer; sample 1 was collected from lymph node and sample 2 was collected from the primary tumor. Patient B was diagnosed with lung cancer NSCLC adenocarcinoma; sample 1 was collected from pleural effusion and sample 2 was collected from solid tumor. Patient C was diagnosed with small cell lung carcinoma; samples 1 and 2 were collected from separate sections of the lung. Ages of the patients ranged from 51-79 years old with an average age of 65 years. One patient was male and two were female.

### Results:

Six paired specimens were obtained from 1 pancreatic islet cell cancer patient, 1 lung adenocarcinoma, and 1 small cell lung carcinoma patient. The mean number of individual drug apoptosis tests analyzed per patient was 34.7 (range 15 to 46). Data obtained using the MiCK assay for these six paired specimens are summarized in Table 4.

### Conclusions:

The data obtained from this study further support that drug-induced apoptosis measured by the MiCK assay in different tumor sites is homogeneous defined as within two (2) standard deviations (SD) as demonstrated in NSCLC (Example 1). This study included tumors from three additional solid tumor cancers: pancreatic islet cell cancer, lung adenocarcinoma and small cell lung carcinoma. Based on these results, coupled with the results from Example 1, use of the MiCK assay can determine and enable recommendation of the most effective drug candidate or combination of drug candidates for an individual patient by testing a single tumor site.

TABLE 1
Enzyme Utilization Dependent Upon Tumor Type of Specimen

| | | Other enzyme |
|---|---|---|
| | First choice enzyme + | possibility + |
| Tumor type | DNase 0.008% | DNase 0.008% |
| Bladder | Collagenase IV 300 U/mL | |
| Breast | Collagenase IV 300 U/mL | Collagenase III |
| | | 200 U/mL |
| Cervix | Trypsin 0.25% | |
| Colon | Collagenase I 300 U/mL + | Trypsin 0.25% |
| | Dispase 1 U/mL | |
| Endometrial | Trypsin 0.25% | — |
| Kidney | Collagenase IV 300 U/mL | — |
| Gastric | Trypsin 0.25% | — |
| Leiomyosarcoma | Trypsin 0.25% | Collagenase IV |
| | | 300 U/mL |
| Liver | Collagenase IV 300 U/mL | |
| Lung | Collagenase IV 300 U/mL | — |
| Melanoma | Collagenase IV 300 U/mL | |
| Ovarian | Trypsin 0.25% | — |
| Pancreas | Collagenase IV 300 U/mL + | — |
| | Hyaluronidase 0.1 U/mL | |
| Prostate | Collagenase I 300 U/mL | — |
| Soft tissue sarcoma | Trypsin 0.25% | — |
| Thymus | Collagenase 1 300 U/mL | — |

TABLE 2
Final Cell Suspension Plating Protocol

| | 96 well plate | | 384 clear plate/ | | 384 black plate/ | |
|---|---|---|---|---|---|---|
| | Corning # 3696 | | Corning # 3701 | | Greiner# 788091 | |
| Pre-fill Medium | 30 | μL | 15 | μL | 2.5 | μL |
| Cell suspension | 90 | μL | 45 | μL | 15 | μL |
| Drug | 5 | μL (25X) | 2.5 | μL (25X) | 2.5 | μL (8X) |
| Oil | 30 | μL | 15 | μL | 7 | μL |

TABLE 3
Results from MiCK Assay performed on NSCLC lung tumors from multiple
sites in an individual patient

| | Tumor | | | | | | | | | |
|---|---|---|---|---|---|---|---|---|---|---|
| | A1 | A2 | B1 | B2 | C1 | C2 | D1 | D2 | E1 | E2 |
| Paclitaxel KU | NA | 1.40 | 1.70 | 0.90 | 0.00 | 1.00 | 1.40 | 0.90 | 0.00 | 0.50 |
| Docetaxel KU | NA | 1.10 | 2.60 | 2.00 | 0.90 | 2.30 | 0.50 | 0.70 | 0.90 | 0.20 |
| 4HI (Ifosfamide) | NA | 3.40 | 2.70 | 0.70 | 0.50 | 0.50 | 1.60 | 1.20 | 0.10 | 0.60 |
| KU | | | | | | | | | | |
| 4HC (Cytoxan) | NA | 3.10 | 1.10 | 1.20 | 0.60 | 0.00 | 1.20 | 1.20 | 0.50 | 0.30 |
| KU | | | | | | | | | | |
| Sunitinib KU | NA | NA | NA | NA | NA | NA | NA | NA | NA | NA |
| Epirubicin KU | NA | 3.80 | 0.90 | 1.20 | NA | NA | 1.40 | NA | NA | NA |
| Sorafenib KU | NA | NA | NA | NA | NA | NA | NA | NA | NA | NA |
| Temo-zolomide/ | NA | NA | NA | NA | NA | NA | NA | NA | NA | NA |
| Xeloda KU | | | | | | | | | | |
| Carbo/Paclitaxel | NA | NA | NA | NA | NA | NA | NA | NA | NA | NA |
| KU | | | | | | | | | | |
| Irinotecan KU | NA | 1.30 | 1.60 | 1.50 | 0.40 | 0.30 | 0.40 | 0.90 | 0.60 | 0.10 |
| Bendamustine KU | NA | NA | NA | NA | NA | NA | NA | NA | NA | NA |
| Cisplatin KU | NA | 2.80 | 1.50 | 2.10 | 0.00 | 2.10 | 1.00 | 1.00 | 0.80 | 0.50 |
| Dacarbazine KU | NA | NA | NA | NA | NA | NA | NA | NA | NA | NA |
| Everolimus KU | NA | NA | NA | NA | NA | NA | NA | NA | NA | NA |
| Doxorubicin KU | NA | 3.80 | 1.70 | 0.90 | 0.80 | 1.50 | 1.50 | 1.40 | 1.60 | 1.50 |
| Idarubicin KU | NA | 4.00 | 1.20 | 1.10 | 0.90 | NA | 1.30 | NA | NA | NA |
| Vinorelbine KU | NA | 2.10 | 1.20 | 1.70 | 0.20 | 0.00 | 0.40 | 1.00 | 0.40 | 0.00 |
| Oxaliplatin KU | NA | NA | NA | NA | NA | NA | NA | NA | NA | NA |
| Gemcitabine KU | NA | 0.70 | 1.20 | 1.10 | 0.00 | 0.70 | 0.10 | 0.90 | 0.30 | 0.50 |
| Carboplatin KU | NA | 1.50 | 1.90 | 2.20 | 0.40 | 1.20 | 1.00 | 0.80 | 0.30 | 0.30 |
| Etoposide KU | NA | 1.20 | 1.10 | 1.20 | 0.80 | 1.70 | 1.60 | 0.90 | 0.40 | 0.00 |
| Mitomycin KU | NA | 1.00 | 1.20 | 1.00 | 0.00 | NA | 0.60 | NA | NA | NA |
| Vidaza KU | NA | NA | NA | NA | NA | NA | NA | NA | NA | NA |
| Vinblastine KU | NA | NA | NA | NA | NA | NA | NA | NA | NA | NA |
| CCNU KU | NA | NA | NA | NA | NA | NA | NA | NA | NA | NA |
| Ixabepilone KU | NA | NA | NA | NA | NA | NA | NA | NA | NA | NA |
| Vincristine KU | NA | 2.80 | 1.30 | 1.50 | 0.00 | 0.40 | 0.40 | 0.90 | 0.10 | 0.10 |
| Carmustine KU | NA | NA | NA | NA | NA | NA | NA | NA | NA | NA |
| Nitrogen Mustard | NA | NA | NA | NA | NA | NA | NA | NA | NA | NA |
| KU | | | | | | | | | | |
| Mitoxantrone KU | NA | NA | NA | NA | NA | NA | NA | NA | NA | NA |
| Velcade KU | NA | 1.10 | 2.00 | 2.00 | NA | NA | 0.60 | NA | NA | NA |
| Abraxane KU | NA | 0.70 | NA | NA | 0.00 | NA | NA | NA | NA | NA |
| Methotrexate KU | NA | 0.50 | 1.00 | NA | NA | NA | NA | NA | NA | NA |
| Tarceva KU | NA | 0.80 | 0.60 | 0.60 | NA | NA | 0.5 | NA | NA | NA |
| Temo-zolomide | NA | NA | NA | NA | NA | NA | NA | NA | NA | NA |
| KU | | | | | | | | | | |
| Cladribine KU | NA | NA | NA | NA | NA | NA | NA | NA | NA | NA |
| Melphalan KU | NA | NA | NA | NA | NA | NA | NA | NA | NA | NA |
| 5FU KU | NA | NA | NA | NA | NA | NA | NA | NA | NA | NA |
| Xeloda KU | NA | NA | NA | NA | NA | NA | NA | NA | NA | NA |
| Gleevec KU | NA | NA | NA | NA | NA | NA | NA | NA | NA | NA |
| Streptozocin KU | NA | NA | NA | NA | NA | NA | NA | NA | NA | NA |
| Alimta KU | NA | 1.20 | 1.40 | 1.60 | 0.80 | NA | 0.70 | 0.60 | NA | NA |
| Dactinomycin KU | NA | 4.70 | 1.20 | 0.90 | NA | NA | 0.90 | NA | NA | NA |
| Alimta/Doxo KU | NA | 4.60 | 1.10 | 0.90 | NA | NA | 1.60 | NA | NA | NA |
| 4HC/Doxo/ | NA | 4.00 | 1.40 | 2.00 | 1.10 | NA | 1.50 | 1.00 | 1.40 | 0.70 |
| Vincristine KU | | | | | | | | | | |
| Daunorubicin KU | NA | 3.80 | 1.40 | 1.40 | NA | NA | NA | NA | NA | NA |
| Topotecan KU | NA | 3.60 | 1.70 | 0.80 | 0.50 | 0.00 | 0.40 | 1.00 | 0.70 | 0.30 |
| CisP/Irinotecan | NA | 3.50 | 1.40 | 0.40 | 1.40 | NA | 1.30 | 1.40 | NA | 0.50 |
| KU | | | | | | | | | | |
| Alimta/CisP KU | NA | 3.10 | 1.00 | 1.50 | 1.20 | NA | 1.30 | 1.00 | NA | NA |
| CisP/Vinblastine | NA | 2.90 | 2.00 | 1.80 | 0.40 | NA | 1.50 | 1.40 | NA | NA |
| KU | | | | | | | | | | |
| Alimta/4HC KU | NA | 2.60 | 1.10 | 0.00 | NA | NA | 0.60 | 0.30 | NA | NA |
| Gemcitabine/CisP | NA | 2.50 | 1.20 | 1.80 | 0.60 | NA | 1.00 | 0.80 | NA | NA |
| KU | | | | | | | | | | |
| Carbo/Irinotecan | NA | 2.50 | 1.20 | 0.70 | 1.40 | NA | 0.70 | 0.60 | NA | 0.30 |
| KU | | | | | | | | | | |
| CisP/Etoposide | 3.20 | 2.50 | 2.10 | 1.40 | 1.70 | NA | 1.20 | 1.30 | 1.00 | 0.50 |
| KU | | | | | | | | | | |
| CisP/Vinorelbine | NA | 2.20 | 1.30 | 1.50 | 0.70 | NA | 1.50 | 1.10 | NA | NA |
| KU | | | | | | | | | | |
| Alimta/Paclitaxel | NA | 1.80 | 0.90 | 0.90 | NA | NA | 0.60 | 0.60 | NA | NA |
| KU | | | | | | | | | | |
| Alimta/Carbo KU | NA | 1.80 | 2.00 | 0.80 | 0.80 | NA | 0.40 | 0.40 | NA | NA |
| Carbo/Etoposide | 0.90 | 1.80 | 1.30 | 1.10 | 0.60 | NA | 0.70 | 1.20 | 0.60 | 0.30 |
| KU | | | | | | | | | | |
| CisP/Docetaxel | NA | 1.60 | 1.50 | 1.90 | 1.20 | NA | 1.20 | 0.90 | NA | NA |
| KU | | | | | | | | | | |
| Alimta/Docetaxel | NA | 1.30 | 2.50 | 1.20 | NA | NA | 0.50 | 0.60 | NA | NA |
| KU | | | | | | | | | | |
| Bleomycin KU | NA | 1.00 | 1.90 | 1.30 | NA | NA | NA | NA | NA | NA |
| Paclitaxel/CisP | 0.40 | 0.70 | 0.20 | 1.00 | 1.30 | 0.10 | 0.90 | 0.70 | 0.40 | 0.10 |
| KU | | | | | | | | | | |
| Alimta/ | NA | 0.60 | 0.90 | 1.8 | NA | NA | 0.60 | 0.30 | NA | NA |
| Gemcitabine KU | | | | | | | | | | |
| Vinor/Docetaxel | NA | NA | NA | NA | NA | NA | NA | NA | NA | NA |
| KU | | | | | | | | | | |
| 5FU/Leuco/ | NA | NA | NA | NA | NA | NA | NA | NA | NA | NA |
| Docetaxel KU | | | | | | | | | | |
| Docetaxel/CisP/ | NA | NA | NA | NA | NA | NA | NA | NA | NA | NA |
| 5FU/Leuco KU | | | | | | | | | | |
| Gemcitabine/ | NA | NA | NA | NA | NA | NA | NA | NA | NA | NA |
| Docetaxel KU | | | | | | | | | | |
| 4HC/Docetaxel | NA | NA | NA | NA | NA | NA | NA | NA | NA | NA |
| KU | | | | | | | | | | |
| Carbo/Docetaxel | NA | NA | NA | NA | NA | NA | NA | NA | NA | NA |
| KU | | | | | | | | | | |
| 4HC/Doxo KU | NA | NA | NA | NA | NA | NA | NA | NA | NA | NA |
| 5FU/Leuco/CisP | NA | NA | NA | NA | NA | NA | NA | NA | NA | NA |
| KU | | | | | | | | | | |
| 5FU/Leuco KU | NA | NA | NA | NA | NA | NA | NA | NA | NA | NA |
| Irinotecan/5FU/ | NA | NA | NA | NA | NA | NA | NA | NA | NA | NA |
| Leuco KU | | | | | | | | | | |
| Epi/CisP/5FU/ | NA | NA | NA | NA | NA | NA | NA | NA | NA | NA |
| Leuco KU | | | | | | | | | | |
| Carbo/Paclitaxel/ | NA | NA | NA | NA | NA | NA | NA | NA | NA | NA |
| Etoposide KU | | | | | | | | | | |
| 5FU/Leuco/Oxali | NA | NA | NA | NA | NA | NA | NA | NA | NA | NA |
| KU | | | | | | | | | | |
| 5FU/Leuco/Carbo | NA | NA | NA | NA | NA | NA | NA | NA | NA | NA |
| KU | | | | | | | | | | |
| Bortezomib KU | NA | NA | NA | NA | 0.20 | NA | NA | NA | NA | NA |
| CisP/5FU/Leuco/ | NA | NA | NA | NA | NA | NA | NA | NA | NA | NA |
| Paclitaxel KU | | | | | | | | | | |
| Doxil KU | NA | NA | NA | NA | NA | NA | NA | NA | NA | NA |
| Pemetrexed KU | NA | NA | NA | NA | NA | NA | NA | NA | NA | NA |
| CisP/Pemetrexed | NA | NA | NA | NA | NA | NA | NA | NA | NA | NA |
| KU | | | | | | | | | | |
| Pemetrexed/ | NA | NA | NA | NA | NA | NA | NA | NA | NA | NA |
| Gemcitabine KU | | | | | | | | | | |
| Pemetrexed/ | NA | NA | NA | NA | NA | NA | NA | NA | NA | NA |
| Paclitaxel KU | | | | | | | | | | |

Tumor A1: Probable mesothelioma (solid tumor);
A2: Probable mesothelioma (pleural effusion);
B1: NSCLC, bilateral well-differentiated adenocarcinoma (site 1A);
B2: NSCLC, bilateral well-differentiated adenocarcinoma (site 1B);
C1: NSCLC, multi-focal, recurrent adenocarcinoma (site A);
C2: NSCLC, multi-focal, recurrent adenocarcinoma (site B);
D1: NSCLC, multi-focal, recurrent adenocarcinoma (right lung);
D2: NSCLC, multi-focal, recurrent adenocarcinoma (lymph node);
E1: NSCLC, multi-focal adenocarcinoma (site A);
E2: NSCLC, multi-focal adenocarcinoma (site B).

TABLE 4
Results from MiCK Assay performed on various tumors from
multiple sites in an individual patient

| | Tumor | | | | | |
|---|---|---|---|---|---|---|
| | A1 | A2 | B1 | B2 | C1 | C2 |
| Paclitaxel KU | 3.43 | 2.06 | 3.00 | 4.00 | 1.70 | 3.60 |
| Docetaxel KU | 3.20 | 2.65 | 4.20 | 2.40 | 3.20 | 2.10 |
| 4HI (Ifosfamide) KU | 3.01 | NA | 2.40 | NA | 1.50 | 1.60 |
| 4HC (Cytoxan) KU | 3.00 | NA | 1.20 | 2.40 | 1.80 | 2.40 |
| Sunitinib KU | 1.93 | 2.06 | 0.70 | NA | NA | NA |
| Epirubicin KU | 1.61 | NA | 2.70 | 3.50 | 8.70 | 3.20 |
| Sorafenib KU | 1.57 | NA | NA | NA | NA | NA |
| Temozolomide/Xeloda KU | 1.50 | 0.00 | NA | NA | NA | NA |
| Carbo/Paclitaxel KU | 1.50 | 4.13 | 4.50 | 2.00 | 0.60 | 1.30 |
| Irinotecan KU | 1.50 | 1.16 | 0.60 | NA | 1.40 | 2.10 |
| Bendamustine KU | 1.39 | NA | NA | NA | NA | NA |
| Cisplatin KU | 1.31 | 1.75 | 2.20 | 2.20 | 6.40 | 5.40 |
| Dacarbazine KU | 1.31 | 0.00 | NA | NA | NA | NA |
| Everolimus KU | 1.31 | 0.00 | NA | NA | 0.50 | 1.00 |
| Doxorubicin KU | 1.27 | 2.06 | 3.50 | 3.50 | 1.60 | 0.90 |
| Idarubicin KU | 1.22 | NA | NA | NA | 8.00 | 3.00 |
| Vinorelbine KU | 1.18 | NA | 3.90 | 2.70 | 3.50 | 2.50 |
| Oxaliplatin KU | 1.08 | NA | 3.00 | 3.10 | 4.40 | 2.50 |
| Gemcitabine KU | 1.08 | NA | 1.10 | 3.20 | 2.20 | 1.30 |
| Carboplatin KU | 1.08 | 1.48 | 1.20 | 2.40 | 5.30 | 2.60 |
| Etoposide KU | 1.08 | 0.00 | 2.30 | NA | 4.60 | 2.40 |
| Mitomycin KU | 1.04 | NA | 0.90 | NA | 6.30 | 4.40 |
| Vidaza KU | 1.00 | NA | NA | NA | NA | NA |
| Vinblastine KU | 1.00 | NA | NA | NA | 3.10 | 2.00 |
| CCNU KU | 1.00 | NA | NA | NA | 0.90 | 0.60 |
| Ixabepilone KU | 1.00 | NA | NA | NA | NA | NA |
| Vincristine KU | 0.82 | NA | 0.90 | NA | 1.00 | 1.60 |
| Carmustine KU | 0.82 | NA | 1.20 | NA | 3.00 | 0.50 |
| Nitrogen Mustard KU | 0.82 | NA | NA | NA | NA | NA |
| Mitoxantrone KU | 0.82 | NA | 2.30 | NA | 1.60 | 0.40 |
| Velcade KU | 0.79 | NA | 3.40 | NA | 1.60 | 1.80 |
| Abraxane KU | 0.79 | NA | NA | NA | 2.20 | 1.50 |
| Methotrexate KU | 0.66 | NA | 1.40 | 1.30 | 1.30 | 0.60 |
| Tarceva KU | 0.66 | NA | NA | NA | 0.00 | 0.70 |
| Temozolomide KU | 0.62 | 1.22 | NA | NA | NA | NA |
| Cladribine KU | 0.61 | NA | NA | NA | NA | NA |
| Melphalan KU | 0.57 | NA | 3.10 | NA | NA | NA |
| 5FU KU | 0.42 | NA | NA | NA | NA | NA |
| Xeloda KU | 0.42 | 0.90 | NA | NA | NA | NA |
| Gleevec KU | 0.39 | NA | NA | NA | 0.00 | 0.80 |
| Streptozocin KU | 0.23 | 0.0 | NA | NA | NA | NA |
| Alimta KU | 0.22 | NA | NA | NA | NA | NA |
| Dactinomycin KU | NA | NA | 2.70 | NA | 7.70 | 3.00 |
| Alimta/Doxo KU | NA | NA | NA | NA | NA | NA |
| 4HC/Doxo/Vincristine KU | NA | NA | NA | NA | 2.90 | 2.20 |
| Daunorubicin KU | NA | NA | NA | NA | 8.10 | 2.00 |
| Topotecan KU | NA | NA | 1.30 | NA | 9.30 | 4.10 |
| CisP/Irinotecan KU | NA | NA | NA | NA | 3.50 | 4.90 |
| Alimta/CisP KU | NA | NA | NA | NA | NA | NA |
| CisP/Vinblastine KU | NA | NA | NA | NA | 6.30 | 5.40 |
| Alimta/4HC KU | NA | NA | NA | NA | NA | NA |
| Gemcitabine/CisP KU | NA | NA | 1.30 | 2.40 | 5.90 | 4.90 |
| Carbo/Irinotecan KU | NA | NA | NA | NA | 0.40 | 2.00 |
| CisP/Etoposide KU | NA | NA | NA | NA | 8.50 | 4.60 |
| CisP/Vinorelbine KU | NA | NA | NA | NA | 5.70 | 4.70 |
| Alimta/Paclitaxel KU | NA | NA | NA | NA | NA | NA |
| Alimta/Carbo KU | NA | NA | NA | NA | NA | NA |
| Carbo/Etoposide KU | NA | NA | NA | NA | 2.90 | 2.80 |
| CisP/Docetaxel KU | NA | NA | NA | NA | 3.50 | 2.60 |
| Alimta/Docetaxel KU | NA | NA | NA | NA | NA | NA |
| Bleomycin KU | NA | NA | NA | NA | NA | NA |
| Paclitaxel/CisP KU | NA | NA | NA | NA | 0.90 | 2.80 |
| Alimta/Gemcitabine KU | NA | NA | NA | NA | NA | NA |
| Vinor/Docetaxel KU | NA | NA | 3.60 | NA | NA | NA |
| 5FU/Leuco/Docetaxel KU | NA | NA | 3.40 | NA | NA | NA |
| Docetaxel/CisP/5FU/Leuco | NA | NA | 3.30 | 2.30 | NA | NA |
| KU | | | | | | |
| Gemcitabine/Docetaxel | NA | NA | 3.30 | 1.30 | NA | NA |
| KU | | | | | | |
| 4HC/Docetaxel KU | NA | NA | 3.10 | NA | NA | NA |
| Carbo/Docetaxel KU | NA | NA | 3.10 | 1.80 | NA | NA |
| 4HC/Doxo KU | NA | NA | 3.00 | NA | NA | NA |
| 5FU/Leuco/CisP KU | NA | NA | 2.30 | NA | NA | NA |
| 5FU/Leuco KU | NA | NA | 1.90 | 4.20 | 1.00 | 1.10 |
| lrinotecan/5FU/Leuco KU | NA | NA | 1.50 | NA | NA | NA |
| Epi/CisP/5FU/Leuco KU | NA | NA | 1.50 | NA | NA | NA |
| Carbo/Paclitaxel/ | NA | NA | 1.20 | NA | NA | NA |
| Etoposide KU | | | | | | |
| 5FU/Leuco/Oxali KU | NA | NA | 1.20 | 3.70 | NA | NA |
| 5FU/Leuco/Carbo KU | NA | NA | 1.00 | NA | NA | NA |
| Bortezomib KU | NA | NA | NA | NA | NA | NA |
| CisP/5FU/Leuco/Paclitaxel | NA | NA | 2.40 | 3.10 | NA | NA |
| KU | | | | | | |
| Doxil KU | NA | NA | NA | NA | 1.20 | 0.60 |
| Pemetrexed KU | NA | NA | NA | NA | 1.40 | 0.70 |
| CisP/Pemetrexed KU | NA | NA | NA | NA | 8.30 | 5.90 |
| Pemetrexed/Gemcitabine | NA | NA | NA | NA | 1.00 | 1.30 |
| KU | | | | | | |
| Pemetrexed/Paclitaxel KU | NA | NA | NA | NA | 0.00 | 0.90 |

Tumor A1: Pancreatic islet cell cancer (lymph node);
A2: Pancreatic islet cell cancer (primary tumor);
B1: Lung adenocarcinoma (pleural effusion);
B2: Lung adenocarcinoma (solid tumor);
C1: Lung small cell carcinoma (site A);
C2: Lung adenocarcinoma (site B).

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A method of using the MiCK assay to determine the most effective drug candidate or combination of drug candidates for an individual patient by testing a single tumor site.

2. The method of claim 1, the method comprising:
a. plating a single-cell suspension of viable cancer cells obtained from at least one tumor site in an individual patient in at least one well of a plate suitable to be read by a spectrophotometer, wherein the cancer cells are in a concentration sufficient to form a monolayer of cells on the bottom of the well,
b. adding at least one drug candidate to the well in an amount sufficient to achieve a target drug candidate concentration,
c. measuring the optical density of the well at a wavelength of approximately 600 nm using a spectrophotometer at selected time intervals for a selected duration of time,
d. determining a kinetic units (KU) value from the optical density and time measurements, and
e. correlating the KU value with an ability of the anti-cancer drug candidate to induce apoptosis in the cancer cells if the KU value is positive, or an inability of the anti-cancer drug candidate to induce apoptosis in the cancer cells if the KU value is not positive.

3. The method of claim 1, wherein the most effective drug candidate or combination of drug candidates identified for an individual patient by testing a single tumor site is also the most effective drug candidate or combination of drug candidates at at least one other tumor site in the patient.

4. A method of determining the most effective drug candidate or combination of drug candidates for an individual patient, wherein a sample from a primary tumor site or a metastasis of the primary tumor site is tested using the MiCK assay, wherein the most effective drug candidate or combination of drug candidates at the primary tumor site or metastasis thereof is indicative of the most effective drug candidate or combination of drug candidates at other metastases of said primary tumor.

5. The method of claim 4, wherein the sample tested is from the primary tumor site.

6. The method of claim 5, wherein the most effective drug candidate or combination of drug candidates determined is indicative of the most effective drug candidate or combination of drug candidates at all metastases of said primary tumor.

7. The method of claim 4, wherein the tested sample is from a metastatic tumor site.

8. The method of claim 7, wherein the most effective drug candidate or combination of drug candidates determined is indicative of the most effective drug candidate or combination of drug candidates at the primary tumor.

9. The method of claim 7, wherein the most effective drug candidate or combination of drug candidates determined is indicative of the most effective drug candidate or combination of drug candidates at all metastases of said primary tumor.

10. The method of claim 4, wherein the sample from a solid tumor.

11. The method of claim 10, wherein the solid tumor is a pancreatic tumor or a lung tumor.

12. The method of claim 11, wherein the lung tumor is selected from the group consisting of a non-small cell lung tumor; a small cell lung tumor; and a lung adenocarcinoma.

13. The method of claim 4, wherein a kinetic unit (KU) value is obtained by analysis of the sample in the presence of a drug candidate or combination of drug candidates, and said KU value is within two standard deviations of the KU value obtained by analysis of a different tumor site in the patient in the presence of the same drug candidate or combination of drug candidates.

14. The method of claim 4, wherein a kinetic unit (KU) value is obtained by analysis of the sample in the presence of a drug candidate or combination of drug candidates, and said KU value is within one standard deviation of the KU value obtained by analysis of a different tumor site in the patient in the presence of the same drug candidate or combination of drug candidates.

15. A method of treating a subject, comprising:
a. plating a single-cell suspension of viable cancer cells obtained from a tumor site in said subject in at least one well of a plate suitable to be read by a spectrophotometer, wherein the cancer cells are in a concentration sufficient to form a monolayer of cells on the bottom of the well,
b. adding at least one drug candidate to the well in an amount sufficient to achieve a target drug candidate concentration,
c. measuring, at selected time intervals for a selected duration of time, the optical density of the well at a wavelength of approximately 600 nm using a spectrophotometer,
d. determining a kinetic units (KU) value from the optical density and time measurements,
e. correlating the KU value with an ability of the at least one drug candidate to induce apoptosis in the cancer cells if the KU value is positive, or an inability of the at least one drug candidate to induce apoptosis in the cancer cells if the KU value is not positive, and
f. informing said subject or a health professional caring for said subject of said at least one drug candidate, based on said KU value.

16. Use of an assay to determine the efficacy of at least one drug candidate or combination of drug candidates against a primary tumor, the method comprising:
a. plating in at least one well of a plate suitable to be read by a spectrophotometer a single-cell suspension of viable cancer cells obtained from at least one metastatic tumor of said primary tumor of an individual patient, wherein the cancer cells are in a concentration sufficient to form a monolayer of cells on the bottom of the well,
b. adding at least one drug candidate or combination of drug candidates to the well in an amount sufficient to achieve a target concentration,
c. measuring the optical density of the well at a wavelength of approximately 600 nm at selected time intervals for a selected duration of time,
d. determining a kinetic units (KU) value from the optical density and time measurements, and
e. correlating the KU value with an ability of the at least one drug candidate or combination of drug candidates to induce apoptosis in the primary tumor if the KU value is positive, or an inability of the at least one drug candidate or combination of drug candidates to induce apoptosis in the primary tumor if the KU value is not positive.

17. Use of an assay to determine the efficacy of at least one drug candidate or combination of drug candidates against a metastatic tumor, the method comprising:
a. plating in at least one well of a plate suitable to be read by a spectrophotometer a single-cell suspension of viable cancer cells obtained from the primary tumor of the metastatic tumor of an individual patient, wherein the cancer cells are in a concentration sufficient to form a monolayer of cells on the bottom of the well,
b. adding at least one drug candidate or combination of drug candidates to the well in an amount sufficient to achieve a target concentration,
c. measuring the optical density of the well at a wavelength of approximately 600 nm at selected time intervals for a selected duration of time,
d. determining a kinetic units (KU) value from the optical density and time measurements, and
e. correlating the KU value with an ability of the at least one drug candidate or combination of drug candidates to induce apoptosis in the metastatic tumor if the KU value is positive, or an inability of the at least one drug candidate or combination of drug candidates to induce apoptosis in the metastatic tumor if the KU value is not positive.
